(19) United States
(12) Patent Application Publication
(10) Pub. No.: US 2009/0149683 A1
(43) Pub. Date: Jun. 11, 2009

(54) Methods for using material from biodiesel production in hydrocarbon production and refining

(75) Inventors: Spencer S. Awbrey, Conroe, TX (US); H. Casey Riney, Montgomery, TX (US)

Correspondence Address: BURLESON COOKE L.L.P., 2040 NORTH LOOP 336 WEST, SUITE 123, CONROE, TX, 77304, US

(73) Assignee: CAM CHEMICAL MANUFACTURING LLC, Houston, TX (US)

(21) Appl. No.: 11/951,064

(22) Filed: Dec. 5, 2007

(51) Int. Cl. C07C 7/20; B08B 9/02; C01B 3/34

(52) U.S. Cl. 585/15; 134/22.14; 48/127.1

(57) ABSTRACT

An oil or gas treating fluid is provided by processing the waste stream from biodiesel production. The waste stream may be used to treat for hydrate formation in hydrocarbon gas streams, to prevent or break oil and water emulsions formed in hydrocarbon streams, to prevent or remove scales in hydrocarbon streams and as an absorption medium for removing unwanted components of gas streams

### BACKGROUND OF THE INVENTION

1. Field of the Invention

The present invention relates to the use in oil and gas production and refining operations of waste product from biodiesel production units. More particularly, in one embodiment, the waste product is used to prevent hydrate formation in oil and gas equipment or hydrocarbon flow streams. In another embodiment, the product is used to facilitate the separation of oil and water emulsions. In another embodiment, the product is used to remove scale from production or refining facilities or to inhibit scale formation in production facilities. In another embodiment, the product is used as an absorption medium for removing carbon or sulfur compounds from gas streams.

2. Description of Related Art

In the oil and gas industry, a wide variety of chemicals are used to prevent gas hydrate formation in flow streams or equipment. Alcohols and glycols are commonly used. Gas hydrates have a cage-like structure of water and small hydrocarbon molecules (primarily methane and ethane); they form above the freezing point of water when the gas is under pressure. For example, methane hydrate can form at temperatures as high as 75° F. when gas pressure is about 3000 psi. Hydrates can plug flow lines or other equipment when natural gas and water are present in a stream at a pressure above the hydrate formation pressure. Hydrates may also form in gas processing or refining operations when a gas stream is cooled below the hydrate temperature at the pressure of the stream.

The use of byproducts from biodiesel production for anti-icing or deicing has been disclosed (U.S. Pat. No. 7,270,768). The byproducts are suggested for use on surfaces. The only application suggested for oil or gas production is use in a well drilling fluid. US Pat. App. No. 20040195150 discloses the use of biodiesels to decrease the viscosity of heavy hydrocarbons.

When oil and water phases are produced in a well, emulsions often form, either in the well or at the surface. It is desirable to demulsify the stream. Surface active agents are normally used, sometimes in combination with heat or solvents. The efficacy of demulsifiers is affected by the composition of the oil and water phases, conditions of demulsification and the presence of co-surfactants and co-solvents.

Hydrocarbons are normally produced with a water phase that contains inorganic salts Most of these salts remain soluble under conditions in a well, but certain salts (for example, calcium sulfate) precipitate to form scales under some conditions in wells. The scales can plug flow paths into the well or tubulars in die well or even surface equipment receiving the scale-forming stream. Scale inhibitors or scale removers are often injected at the surface of wells or downhole near the point of entry of fluids into a well. Alternatively, capillary tubing may be placed in the well to carry an inhibiting or treating fluid for scales to a selected depth in a well.

Gas streams often contain sulfur or carbon-oxygen compounds. The streams must be treated to remove or partially remove these compounds before sale or further processing of the gas. Liquids are commonly used as absorbents for this removal process.

Recently, the use of biodiesel materials, such as methyl esters of fatty acids derived from either vegetable or animal fats, has become much more common to augment the diesel fuel supply in the United States. Methods of manufacturing biodiesel include breaking the fatty acids free from glycerol. Methods are described in U.S. Pat. Nos. 6,399,800; 6,348,074; 6,015,440; 6,203,585; 6,174,501; and 6,235,104. All these patents are hereby incorporated by reference herein. In all cases, a waste product, made up of glycerin, water, salts of fatty acids and other polar compounds, is produced at the rate of 10 to 25 pounds for every 100 pounds of biodiesel produced by commonly used methods. This large volume of waste product has created a disposal problem and also created a need to find useful applications for the waste stream. In some cases, this product is refined to technical grade or USP grade glycerin. Production of biodiesel is important to reduce dependence on fossil hydrocarbons, and providing the means for achieving value from this waste stream is an important factor in achieving acceptable economics for biodiesel production.

There exists a need in the art for a new low-cost material that can he used either alone or in combination with prior art agents to eliminate the need for or reduce the cost of chemicals presently used for preventing or treating hydrates, scales and emulsions in hydrocarbon streams. There also exists a need for a lower-cost absorption medium for removing unwanted components from a hydrocarbon stream.

### SUMMARY OF INVENTION

An effective amount of a liquid phase, which is referred to herein as an “oil or gas treating fluid” and which is separated from a waste stream from biodiesel production, is added to a hydrocarbon gas stream to prevent formation of or remove gas hydrates in the stream. The treating fluid may be adjusted in pH or water content may be adjusted before use. The treating fluid may alternatively be used to prevent emulsion formation or demulsify oil and water in oil and gas production, gas processing or refining operations. In another embodiment, the treating fluid is used to prevent scale formation or to remove scale formed in oil or gas production and pipeline operations. In yet another embodiment, the fluid is used as an absorption medium in oil or gas production operations.

### DETAILED DESCRIPTION

Processes for production of biodiesel front vegetable or animal oils are well known. As described in U.S. Pat. No. 7,270,768, for example, which is hereby incorporated by reference herein, a hydrolysis process produces a byproduct stream that is relatively high in glycerol content, which is the bottoms stream from a reactor. The overhead stream contains most of the fatty acids.

The high-glycerol (or bottoms) waste stream of a biodiesel process may be used to produce the fluid material disclosed herein, which may be used in oil or gas operations discussed above and which will be referred to herein as an “oil or gas treating fluid.” The oil or gas treating fluid may be prepared by loading a heated mixing tank with the biodiesel byproduct (bottoms) stream, adding acid, preferably hydrochloric acid, to reduce the pH of the biodiesel byproduct stream below about 4.5, then heating the stream to about 150° F. while mixing. The treated waste stream may then be transferred to a separator for phase separation. The top layer will be primarily crude fatty acids, which may be dark in color. The top layer can be as much as 60% of the byproduct stream. The lower layer is the oil or gas treating fluid. Glycerin and other polar compounds are present in this fluid. It may also be dark in color, but color is not important for the uses disclosed herein. After separation, the pH of the oil or gas treating fluid may be adjusted to 7 or higher with sodium hydroxide, potassium hydroxide, a soluble amine or an ethanol-type amine, for example. Ethanol amines are preferred. Water content of the oil or gas treating fluid may be adjusted. For example, water content may be increased to 20 to 30% to lower viscosity. This water content lowers the viscosity to between 14 and 34 cp. (The viscosity of the crude stream may be too high for many pumping conditions in the oil and gas field.) The freezing point of the liquid is normally between −39 and −20° C. and the density is reduced to about. 1.19 at 20° C. in this range of water content. Alternatively, water content may be lowered.

To prevent formation of gas hydrates in produced natural gas streams, it is known to inject anti-freezing compounds such as alcohol or glycol. These compounds are often injected into the gas stream at the well head. They may be injected at the bottom of a well or at any point in a stream where needed. Using similar techniques, the oil or gas treating fluid disclosed above may be injected into a gas stream to prevent formation of gas hydrate in the stream or in equipment carrying the stream, using the type of equipment normally used for injection of alcohols or glycols. An effective amount of the product will be in the range from about 0.1 percent to 70 percent by volume of the stream. Alternatively, the oil or gas treating fluid may be used to prevent formation of gas hydrate in oil and gas production and pipeline valves. The treating fluid may be adjusted to a pH of 4 to 6, for example, and a density of about 1.18. An acrylate polymer such as Carbopol EZ-2 may be added to the fluid at a concentration in the range of about 0.1 to about 0.5% by weight. Graphite or other solid lubricant may be mixed with the liquid at a concentrations range of 2 to 3% by weight. An effective amount of a corrosion inhibitor may be added, along with an ethanol amine to thicken the liquid into a gel or grease. This gel or grease may be placed into a valve by means of the grease port, using well known procedures.

Emulsions of oil and water form in many oil production operations, it is necessary to separate the oil and water before sale of the oil. The separation often occurs in gravity separators and Is aided by addition of demulsifiers, which are surface active chemicals, to the stream. The oil or gas treating fluid disclosed above may be used to increase the effectiveness of demulsifiers or as a substitute for demulsifiers. An effective amount of the oil or gas treating fluid will normally be in the range from about 0.01 percent to about 5 percent by volume. The density of the oil or gas treating fluid, which may be greater than the density of the water phase of the emulsion, may also increase the rate of gravity separation by increasing the density of the water phase. In some cases, the oil or gas treating fluid may be sufficient to demulsify the oil and water without the necessity of adding additional demulsifier. The waste stream may also be added to the oil or water phase before formation of the emulsion, to prevent or decrease the amount of emulsion formed. In addition, the oil or gas treating fluid may also serve as a defoamer for the oil or water phase.

Scales of inorganic or organic materials may form on surfaces during production of oil and gas. The inorganic materials are initially present in a water phase present with the hydrocarbons. There is a need for methods and materials to prevent the deposition of scales and to remove scales that have been formed. Scale prevention is aided by addition of scale inhibitors, including phosphonates and surface active polymers to the stream. The oil or gas treating fluid disclosed herein may be used to increase the effectiveness of these scale inhibitors. An effective amount of the oil or gas treating fluid will normally be in the range from about 0.01 percent to 10 percent by volume. The oil or gas treating fluid disclosed herein may be added to fluid streams in oil and gas production to inhibit formation of scales. Alternatively, the oil or gas treating fluid may be used to remove scales that have formed on surfaces. For example, calcium sulfide is much more soluble in the oil or gas treating fluid disclosed herein than and water or brine. Calcium sulfate is soluble to about 15 parts per 100 parts oil or gas treating fluid. Sulfur is also much more soluble in the oil or gas treating fluid than in water. The oil or gas treating fluid may be injected into flow lines, equipment or wells to remove scale. When the oil or gas treating fluid is used for cleaning, the material may be used after it is made acidic and separated without adjustment of pH. The material may be diluted with water and an acid corrosion inhibitor may be added. Phosphoric acid may be added to the oil or gas treating fluid and other proprietary products used for cleaning, may also be added. The mixture may be diluted with water and circulated through a pipe or other equipment or it may be used in front of a pipeline pig through a pipeline or flow stream. An effective amount of the oil or gas treating fluid will be in the range from 01 percent to about 10 percent.

In another embodiment, the oil or gas treating fluid disclosed herein may be used as an absorption liquid to remove carbon dioxide, carbon monoxide or sulfur compounds from hydrocarbon streams. The oil or gas treating fluid has a high concentration of glycerin, which is a suitable material for absorbing the impurities in hydrocarbon streams. Carbon dioxide and hydrogen sulfide have an effective amount of solubility in the oil or gas treating fluid to be removed using conventional absorption equipment. Thiols or mercaptans can be effectively removed using the oil or gas treating fluid and conventional absorption equipment. The effectiveness of the oil or gas treating fluid may be increased by adding ethanol amines such as monoethanol amine, diethanol amine or triethanolamine to the byproduct stream for pH adjustment. Water content may be adjusted to select an effective concentration.

Although the present invention has been described with reference to specific details, it is not intended that such details should be regarded as limitations on the scope of the invention, except to the extent that they are included in the accompanying claims.

We claim:

1. A method for preventing formation of gas hydrate in a hydrocarbon gas stream, comprising:
adding an effective amount of oil or gas treating fluid separated from a biodiesel byproduct stream to the hydrocarbon gas stream.

2. The method of claim 1 further comprising adjusting pH of the treating fluid to a value greater than about 7 before adding the treating fluid to the hydrocarbon gas stream.

3. The method of claim 1 further comprising adjusting water content of the treating fluid before adding the treating fluid to the hydrocarbon gas stream.

4. A method for demulsifying or preventing oil and water emulsions, comprising:
adding an effective amount of oil or gas treating fluid separated from a biodiesel byproduct stream to the emulsion or to a stream that forms the emulsion.

5. The method of claim 4 further comprising adding a surface active chemical to the emulsion or the stream that forms the emulsion.

6. The method of claim 4 further comprising adjusting the pH of the treating fluid to a value greater than about 7 before adding the treating fluid to the emulsion or the stream that forms the emulsion.

7. The method of claim 4 further comprising adjusting water content of the treating fluid before adding the treating fluid to the emulsion of to a stream that forms the emulsion.

8. A method for preventing scale formation in a hydrocarbon stream, comprising:
adding an effective amount of oil or gas treating fluid separated from a biodiesel byproduct stream to the hydrocarbon stream.

9. The method of claim 8 further comprising adjusting pH of the treating fluid to a value greater than about 7 before adding the treating fluid to the hydrocarbon stream.

10. The method of claim 8 further comprising adjusting water content of the treating fluid before adding the treating fluid to the hydrocarbon gas stream.

11. A method for removing scale from a pipe or apparatus containing a hydrocarbon stream, comprising:
injecting an effective amount of oil or gas treating fluid separated from a biodiesel byproduct stream into the pipe or apparatus so as to contact the scale and removing the treating fluid from the pipe or apparatus.

12. The method of claim 11 further comprising adjusting pH of the treating fluid to a value greater than about 7 before injecting the fluid into the pipe or apparatus.

13. The method of claim 11 further comprising adjusting water content of the treating fluid before injecting the fluid into the pipe or apparatus.

14. A method for removing carbon or sulfur compounds from a gas stream, comprising:
contacting the gas stream with an oil or gas treating fluid separated from a biodiesel byproduct stream and separating the treating fluid from the gas stream.

15. The method of claim 15 further comprising adjusting pH of the treating fluid to a value greater than about 7 before contacting the gas stream with the treating fluid.

16. The method of claim 16 wherein adjusting the pH to a value greater than about 7 is provided by adding an amine to the treating fluid.

17. The method of claim 15 further comprising adjusting water content of the treating fluid before contacting the gas stream with the treating fluid.
